(19) United States
(12) Patent Application Publication
(10) Pub. No.: US 2006/0171843 A1
(43) Pub. Date: Aug. 3, 2006

(54) Sanitizing device and method of sanitizing

(76) Inventor: Dan E. Spears, Brandon, FL (US)

Correspondence Address: Dan E. Spears, 3515 Westfield Drive, Brandon, FL, 33511, US

(21) Appl. No.: 11/050,134

(22) Filed: Feb. 3, 2005

(51) Int. Cl. A61L 2/24; A61L 2/18

(52) U.S. Cl. 422/28; 422/292; 422/3

(57) ABSTRACT

A sanitizing device has a supply of antibacterial fluid capable of rendering at least a portion of bacteria on an object inert. The fluid is sprayed in a path of distribution in a mist while the object is positioned in the path of distribution. Means are provided to propel the fluid, means are provided to activate commencement of the distribution of the fluid and means are provided to terminate the distribution. Preferably when hands of a person are being sanitized means are provided to ensure that the user is a non-child person. This may be facilitated in various ways including sensory measurement of a height of the user, measurement of a weight of the user and measurement of elevational placement of a trunk of the user via heat sensing of the trunk. Preferably suction is provided to remove excess sprayed fluid from about the sanitizing device.

### BACKGROUND

1. Field of the Invention

Generally, the invention relates to rendering bacteria and other microscopic organisms inert on objects. More specifically, the invention relates to devices which sanitize objects by spraying an antibacterial fluid onto the objects.

2. Description of the Prior Art

Numerous methods exist to sanitized objects to kill bacteria and other microscopic organisms residing thereon. When the object is a physical object it may be sanitized in many manners including by heating the object to a relatively high temperature, by placing fluid heated to a relatively high temperature on the object or by placing the object in fluid heated to a relatively high temperature, by subjecting the object to light sources having certain characteristics, such as ultraviolet light, by placing fluids having bacteria killing properties on the object or placing the object in such fluids. When the object to be sanitized is a portion of a living entity various sanitizing methods may be employed including washing with certain soaps and placing certain fluids having bacteria killing properties on the area(s) to be sanitized. Many fluids are known in the art which have bacteria killing properties, such as bleach and alcohol. Certain of these fluids may safely be applied to living tissue for durations sufficient to kill bacteria without adverse effects to the living tissue being treated.

Bacteria refers to a broad class of living entities which exist in nature and which reside on nearly all objects. The term bacteria as used herein refers generally to microscopic organisms of any scientifically defined class. Certain bacteria are harmful to living entities and may cause adverse effects, including illness, disease and infections.

All persons benefit from maintaining a cleanliness during their routine daily activities. This includes cleaning their hands to eliminate or reduce bacteria residing thereon. People handle many objects during routine activities which provide ample opportunity to transfer bacteria and other germs from the objects to their hands. The most common method of reducing or eliminating bacteria on hands involves washing the hands with soap and water. While this method significantly reduces the quantity of bacteria residing on the hands it requires access to running water to provide for convenient washing where the hands may be rinsed following a soaping and washing action. Often people will hastily wash there hands with soap, sometimes merely rinsing their hands without use of soap, where substantial quantities of living bacteria remain following the washing procedure.

Many persons have occupations where they come into contact with other persons or where they handle objects which subsequently are used by other persons or which are used in the handling of things which later come into contact with other persons or which are consumed by other persons. These persons routinely clean their hands prior to performing tasks associated with their occupations.

Cruise ships have been particularly exposed recently to widespread illness amongst persons traveling thereon. For some reason when persons are contained, even in the very large regions of modern cruise ships, for periods of time they are particularly prone to spreading and catching illness. Often when illness breaks out during such cruises the entire cruise will be canceled for all passengers, both those sick and those unaffected, with the ship returning to a port. Cruise ship lines have sustained financial losses as a result of such spread of illness and would benefit from each person on the ship disinfecting their hands at reasonable intervals to prevent or limit spread of germs amongst the passengers. Many other locations exist, such as restaurants, buffet lines, grocery stores, dental and medical offices and libraries amongst others, which would benefit from providing for disinfection of hands by persons before or after engaging in various activities.

It is known in the art to apply alcohol, and other fluids, to living tissue, or non living material, to kill bacteria residing thereon. Certain compositions of matter in fluid form, such as alcohol, have the desired bacteria killing properties but are flammable. Certain compositions of matter in fluid form have the desired bacteria killing properties and are not flammable.

Various hand washing or sanitizing devices are known in the art to sanitized the hands or a user. Typically these devices fall into two classes. The first class involves complicated devices which have structures which mechanically scrub the hands with fluids of various compositions. The second class involves simple devices which merely dispense fluid materials which are then rubbed into the hands by the user.

Various deficiencies exist with conventionally known devices which sanitize, or assist in sanitizing, the hands of the user. As can be seen various attempts have been made to provide a simple method for a person to sanitize his or her hands to eliminate, or significantly reduce, bacteria residing thereon. These attempts have been less efficient than desired. As such, it may be appreciated that there continues to be a need for a simple device to spray antibacterial fluid capable of killing bacteria onto objects, including hands of persons. The present invention substantially fulfills these needs.

### SUMMARY

In view of the foregoing disadvantages inherent in the known methods of sanitizing objects, including hands of persons, your applicant has devised a sanitizing device, and method of use, to provide for rendering at least of portion of bacteria on a portion of an object, such as hands of a user, inert. The sanitizing device has a supply of antibacterial fluid capable of killing bacteria when brought into contact with the bacteria, a distribution nozzle, having a path of distribution, means to propel the antibacterial fluid through the distribution nozzle, activation means to commence distribution of the antibacterial fluid through the distribution nozzle in the path of distribution and means to terminate distribution of the antibacterial fluid through the distribution nozzle.

My invention resides not in any one of these features per se, but rather in the particular combinations of them herein disclosed and it is distinguished from the prior art in these particular combinations of these structures for the functions specified.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto. Those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore a primary object of the present invention to provide for a device which may provide for rapid sanitation of objects, including hands of persons, by spraying antibacterial fluid onto the object.

Other objects include;

a) to provide for a distribution nozzle to spray antibacterial fluid in a path of distribution where the antibacterial fluid is widely distributed in a fine mist to thoroughly coat the surfaces of the object being treated.

b) to provide for a pressurized system having a rapid opening and closing value where a discharge velocity of the antibacterial fluid remains generally uniform during an entire period of discharge.

c) to provide for various methods to activate commencement of the distribution of the antibacterial fluid.

d) to provide for a timing of a duration of discharge of the distribution of the antibacterial fluid where the antibacterial fluid is sprayed for a predetermined period of time.

e) to provide for suction to draw in the antibacterial fluid not impinging the object and remaining thereon where antibacterial fluid is prevented from leaving a sanitizing area of the device.

f) to provide for a determination that a user of the device to have their hands sanitized is a non-child person to prevent children from activating distribution of the antibacterial fluid in the absence of adult supervision.

g) to provide for the antibacterial fluid to be noncombustible to eliminate danger typically associated with the spraying of combustible fluids.

h) to provide for opposing distribution nozzles to cooperate in spraying antibacterial fluid onto opposing sides of an object, including hands of a person.

i) to provide for an air blower to dry the object, including hands of a person, being sanitized following termination of the spraying of the antibacterial fluid onto the object.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated the preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein;

FIG. 1 is a perspective view of a sanitizing device.

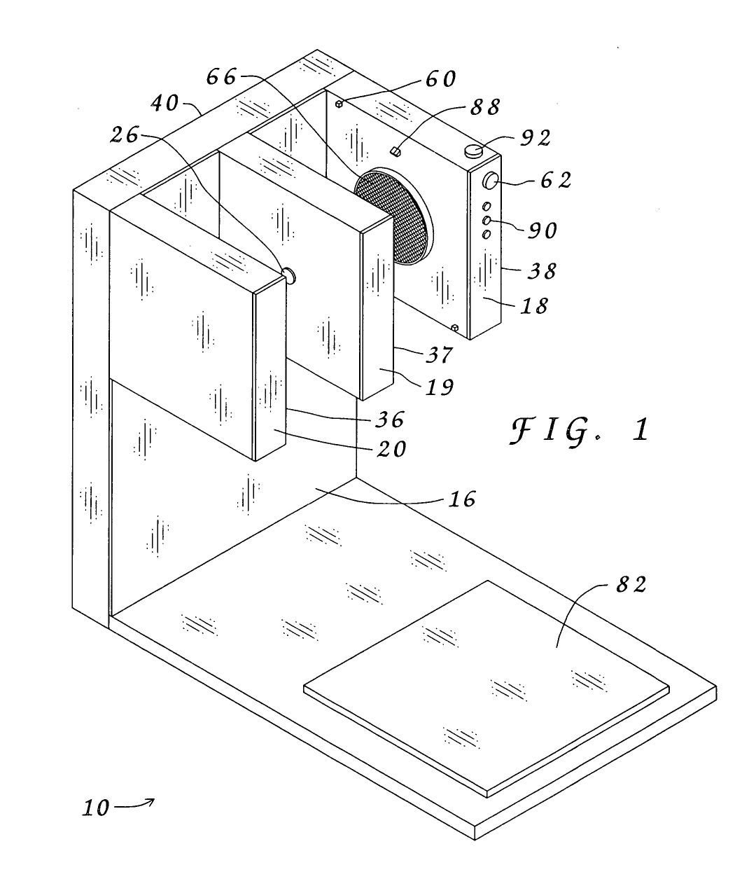

FIG. 2 is an elevational view of the sanitizing device shown in FIG. 1 with various components removed.

### DESCRIPTION

Many different devices having features of the present invention are possible. The following description describes the preferred embodiment of select features of those devices and various combinations thereof. These features may be deployed in various combinations to arrive at various desired working configurations of devices.

Reference is hereafter made to the drawings where like reference numerals refer to like parts throughout the various views.

Various objects exist which have bacteria thereon and which it is often desired to sanitize to render at least of a portion of the bacteria thereon inert. The objects can be merely objects, such as equipment which is routinely handled by persons, or may be portions of a living entity, such as hands of a person. While sanitizing devices having features of the present invention are well suited to sanitize inanimate objects they are extraordinary when utilized to sanitize hands of persons.

People often handle objects or interact by touching other people during normal activities of daily living. Many examples exist where bacteria, germs, viruses and other microscopic living organisms exists which routinely reach hands of persons. All of these microscopic living organisms are generically referred to herein as bacteria. Various infectious diseases, including variations of hepatitis, may be spread by incidental contact in the absence of adequate sanitizing procedures. Several examples of activities which provide opportunity for collection of bacteria to hands include shaking hands, handling mail or other papers and touching or petting animals, such as dogs or cats.

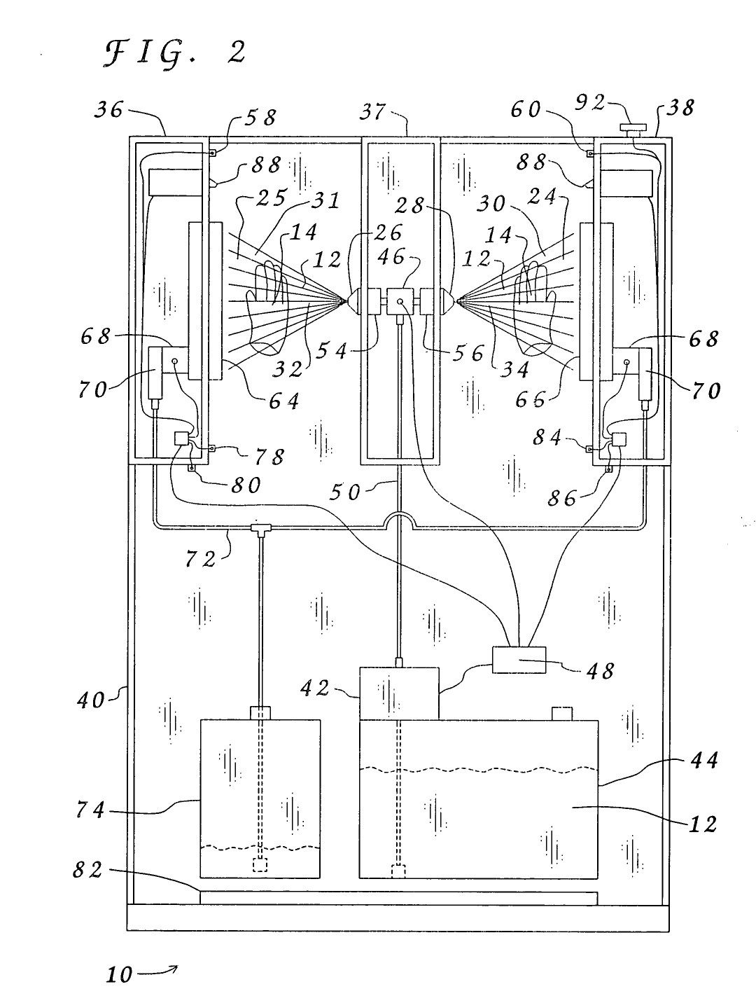

A sanitizing device 10 is depicted in the various views capable of spraying an antibacterial fluid 12 to sanitize hands 14. Sanitizing device 10 provides for rendering at least of portion of bacteria on a portion, hands 14, of an object, a person, inert. Antibacterial fluid 12, as known in the art, is capable of killing bacteria when brought into contact with the bacteria. Preferably, hands 14 are rubbed together following placement of antibacterial fluid 12 thereon. Sanitizing device 10 is depicted in FIG. 2 with a back panel 16, a first extension panel 18, a center extension panel 19 and a second extension panel 20 removed for clarity of understanding of the components concealed therebehind. Preferably, sanitizing device 10 will provide for sanitizing of hands 14 while the user does not physically touch any portion of sanitizing device 10.

Sanitizing device 10 is depicted as a standing unit which rests upon the floor and which users approach and use. Sanitizing devices having features of the present invention may have various configurations depending upon desired usage. Examples of such configurations include table top units, wall mounted units and hanging units such as mounted under a cabinet. Other examples involve transportable units which may be configured for exchange between a deployed orientation and a much more compact transport orientation which may be readily and conveniently transported between usage locations. Still other examples involves mobile units which may be positioned on a transport vehicle, such as police cars, fire trucks, ambulances, taxis, airplanes, buses, trains amongst others. In the field of automobiles, including police vehicles, it is possible to position the sanitizing device on the vehicle where persons may sanitize their hands conveniently. When the vehicle is a police vehicle the sanitizing device may be positioned inside or outside of the vehicle including in the light bar assembly typically located on the roof of the vehicle or in the truck. Very small sanitizing devices are envisioned which take up very little room when deployed. Such devices may be battery powered and very transportable. It is desired that the sanitizing device, in any embodiment, be attractive and have aesthetic appeal. This is particularly true when the sanitizing device is provided by a business entity for use by their patrons.

Opposing nozzles are depicted for simultaneous spraying of both hands of a person at the same time. Preferably the sprayed antibacterial fluid is directed to palms of the hands which typically are used by persons handling objects. Ideally, the user would flex and spread their fingers during the sanitizing procedure to ensure that antibacterial fluid reaches as much skin as possible. Flexing of the fingers also tends to bring the fingertips into the line of misting of the antibacterial fluid where the area under the fingernails are exposed to direct contact by the antibacterial fluid being sprayed. Preferably, the spraying of the antibacterial fluid in the mist is of sufficient density and duration to provide for a coating of at least the palms and inside surfaces of the fingers of the hands so that bacteria in the ridges of the prints are contacted by the antibacterial fluid to render that bacteria inert. Variations are possible where opposing inwardly directed nozzles converge to spray both side of a hand simultaneously with each hand being passed through the device separately. It is possible to provide a single spray nozzle with user manipulation of their hands to disperse the antibacterial fluid to all surfaces of the hands. Blow dryers for washed hands are well known in the art. It is possible to provide for antibacterial fluid discharge within the air flow of such devices where hands may be sanitized and dried in a single quick convenient procedure. In any of the variations of embodiments of devices it is a strong desire to prevent accidental exposure of children to antibacterial fluid being sprayed. Therefore, direction of discharge should always be considered when configuring a specific embodiment or when installing or using a specific embodiment. In certain embodiments it is possible to provide blocking surfaces, such as shelves or panels, which prevent discharged antibacterial fluid from entering areas where children may be, such as about floor areas. Such panels or shelves may serve a dual purpose of holding associated items, such as towels or literature. Preferably, instructions or other information will be provided to users regarding operation of the sanitizing device. Such information may be in printed form or may be displayed electronically, such as text on a display or video on a display. Various advertisement material, in electronic or print form, may also be provided for viewing during the sanitizing procedure.

Sanitizing device 10 sprays antibacterial fluid 12 in a first mist 24 through a first distribution nozzle 26 and in a second mist 25 through a second distribution nozzle 28 into a first sanitizing area 30 and a second sanitizing area 31 respectively. Of course a single nozzle may be deployed if desired. Many different types of nozzles, as conventionally known in the art, may be used with the present invention. First distribution nozzle 26 has a path of distribution 32 while second distribution nozzle 28 has a path of distribution 34. First distribution nozzle 26 is positioned on a center extension 37. Second distribution nozzle 28 is also positioned on center extension 37 opposing first distribution nozzle 26. If desired, center extension 37 may be displaceable to provide for a desired spacial positioning including being pivotal relative to a back member 40. Such adjustable capabilities permit placement of nozzles 26 and 28 depending upon specific needs during usage.

Means to propel antibacterial fluid 12 through nozzles 26 and 28 will be provided. A pump 42 draws antibacterial fluid 12 from a storage tank 44 and transfers it under pressure to a branch valve 46. Branch valve 46 has a valve, conventionally known in the art and not shown, contained therein which is controlled by a control panel 48. Branch valve 46 selectively releases antibacterial fluid 12 under pressure to first distribution nozzle 26 and second distribution nozzle 28. Control panel 48 also provides operational control over pump 42. Preferably fluid pressure remains within spray conduit 50 when branch valve 46 is not being actively instructed to discharge antibacterial fluid 12 through nozzles 26 and 28. While pressurized movement of antibacterial fluid 12 is preferred, an air discharge with draw of antibacterial fluid 12 is envisioned.

While control panel 48 is depicted as providing centralized control over various components it is possible to provide localized control to the various components if desired. A first high pressure valve 54 and a second high pressure valve 56 maintain pressure within spray conduit 50 while preventing any discharge of antibacterial fluid 12 through nozzles 26 and 28. When control panel 48 causes branch valve 46 to release antibacterial fluid 12 to spray conduit 50 high pressure valves 54 and 56 open and antibacterial fluid 12 is discharged through nozzles 26 and 28. When control panel 48 causes branch valve 46 to terminate release of antibacterial fluid 12 high pressure valves 54 and 56 close and antibacterial fluid 12 stops being discharged through nozzles 26 and 28. High pressure valves 54 and 56 provide for a pressurized system wherein high pressure valves 54 and 56 provide for a rapid closure wherein a discharge velocity of antibacterial fluid 12 remains generally uniform during an entire period of discharge between the commencement of distribution and the termination of distribution. Various types of high pressure valves 54 and 56 are known in the art and may be employed with the present invention. This arrangement eliminated dripping or trickling of antibacterial fluid 12 at the beginning of spraying and at the end of spraying.

Activation means provides for commencement of distribution of antibacterial fluid 12 through nozzles 26 and 28. This provides for hands 14 to have antibacterial fluid 12 sprayed thereon to kill at least a portion of bacteria on hands 14. Various methods may be utilized to provide the required activation means. Sensors 58 and 60 are linked to communicate with control panel 48 and have means, such as motion detection or proximity detection as known in the art, to determine a presence of one of the hands 14 within sanitizing areas 30 or 31. Such detection by sensors 58 and 60 causing control panel 48 to commence distribution of antibacterial fluid 12 to sanitize hands 14. A pressure activated switch 62 may be manually engaged by the user to activate distribution of antibacterial fluid 12 to sanitize hands 14. Preferably, control panel 48 has delay means, as known in the art, to provide for a delay of commencement of distribution of antibacterial fluid 12 through nozzles 26 and 28 a predetermined interval of time following manual engagement of pressure activated switch 62 by the user. This provides for the user to position hands 14 relative to sanitizing areas 30 and 31.

Means are provided to terminate distribution of antibacterial fluid 12 through nozzles 26 and 28. Various methods may be utilized to provide the required termination means. Timing means, controlled by control panel 48, terminates distribution of antibacterial fluid 12 through nozzles 26 and 28 at a predetermined interval of time following the commencement of the distribution of antibacterial fluid 12 through nozzles 26 and 28. Alternatively, sensors 58 and 60 may be utilized to cause control panel 48 to terminate distribution of antibacterial fluid 12 to sanitize hands 14.

Suction means provides for a drawing in of at least a portion of antibacterial fluid 12 propelled through nozzles 26 and 28 to prevent antibacterial fluid 12 from leaving sanitizing areas 30 and 31. A first inlet manifold 64 is positioned on a first extension 36 while a second inlet manifold 66 is positioned on a second extension 38. Each inlet manifold 64 and 66 is in communication with a suction fan 68 which draw air, and antibacterial fluid 12, through manifolds 64 and 66 respectively. A condensing member 70 separates antibacterial fluid 12 following passage through each suction fan 68. Antibacterial fluid 12 then moves through a return conduit 72 and is dispersed into a waste tank 74. Control panel 48 provides control over suction fans 68 which preferably operate for a predetermined period of time following termination of discharge of antibacterial fluid 12 into sanitizing areas 30 and 31.

It is a strong desire to prevent young children from activating release of antibacterial fluid without adult supervision. Various means may be employed to provide for this prevention. Various sensory determinations may be utilized to fulfill this desire to ensure that the user is a non-child. The term blocking means refers to preventing activation of the distribution of antibacterial fluid by the sanitizing device having features of the present invention.

A sensor 78 is positioned at an elevational height sufficient to ensure that the user has a height at least of a minimal measurement to ensure that the user is the non-child person. Control panel 48 provides the blocking means to prevent the commencement of distribution of antibacterial fluid 12 through nozzles 26 and 28 in the absence of sensor 78 determining the presence of the non-child person as the user. A sensor 80, having heat detecting means and capable of determining a presence of a heat source at a predetermined location relative to sanitizing areas 30 and 31, ensures that a trunk, not shown, of the user is positioned at a height at least of a minimal measurement to ensure that the user is the non-child person. A weight sensor 82, in mat form which the user stands upon, having weight determining means ensures that the user is of a minimal weight to ensure that the user is the non-child person.

It is preferred to use an antibacterial fluid which is not combustible with sanitizing device of the present invention for obvious safety reasons. When combustible fluid is used it is preferred to provide various safety features to reduce the likelihood of combustion and to contain and extinguish such combustion in the event of ignition.

A first class of safety features detect potential problems and prevent operation of discharge of fluid from the device. A second class of safety features detect actual problems which are occurring and terminate discharge of fluid from the device. A third class of safety features detect actual combustion occurring and activate fire suppression features to extinguish such combustion. Regarding detection of potential problems various sensors may be deployed which monitor for smoke, scan for thermal sources in a range which might cause ignition, scan for radio interference, monitor for power spikes and voltage ranges from the power source or within the system and monitor for frequency changes in operational power, amongst others. A sensor 84, having at least one of the desired sensing capabilities, monitors for such potential problems, and when such a potential problem is identified, control panel 48 would not be capable of sending a signal to discharge antibacterial fluid 12 through nozzles 26 and 28. Regarding detection of an actual existing problem by sensor 84 during discharge of antibacterial fluid 12 through nozzles 26 and 28 control panel 48 would terminate discharge of antibacterial fluid 12. A sensor 86 is capable of detection of combustion actively occurring and causes, directly or through control panel 48, activation of a fire suppression system 88, as known in the art, to extinguish such combustion.

A series of indicator lights 90 provide information regarding operational condition of sanitizing device 10 to the use. It is preferred to provide an override switch 92 which, when manually activated, permits user override of certain of the safety lockouts mentioned above where the user may continue to operate sanitizing device 10 despite detection of certain potential problems.

With respect to the above description then, it is to be realized that the optimum dimensional relationships for the parts of the invention, to include variations in size, material, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A sanitizing device to provide for rendering at least of portion of bacteria on a portion of an object inert, the sanitizing device comprising:
a) a supply of antibacterial fluid capable of killing bacteria when brought into contact with the bacteria;
b) a distribution nozzle having a path of distribution;
c) means to propel the antibacterial fluid through the distribution nozzle;
d) activation means to commence distribution of the antibacterial fluid through the distribution nozzle in the path of distribution wherein the portion of the object may have the antibacterial fluid sprayed thereon to kill at least a portion of bacteria on the portion of the object;
e) means to terminate distribution of the antibacterial fluid through the distribution nozzle.

2. The sanitizing device defined in claim 1 wherein the object is a person and wherein the portion of the object is a hand of the person.

3. The sanitizing device defined in claim 2 further comprises means to determine a presence of the hand of the user in the path of distribution of the distribution nozzle and wherein the activation means is dependent upon the hand of the user being present in the path of distribution of the distribution nozzle.

4. The sanitizing device defined in claim 1 further comprising a second distribution nozzle having a path of distribution and wherein the path of distribution of the distribution nozzle is away from the path of distribution of the second distribution nozzle.

5. The sanitizing device defined in claim 1 further comprising suction means to draw in at least a portion of the antibacterial fluid propelled through the distribution nozzle wherein the antibacterial fluid is generally prevented from leaving a sanitizing area of the sanitizing device.

6. The sanitizing device defined in claim 1 wherein the means to terminate distribution of the antibacterial fluid through the distribution nozzle further comprises timing means to terminate distribution of the antibacterial fluid through the distribution nozzle at a predetermined interval of time following the commencement of the distribution of the antibacterial fluid through the distribution nozzle.

7. The sanitizing device defined in claim 1 wherein the means to propel the antibacterial fluid through the distribution nozzle further comprises a pressurized system having a rapid closure valve wherein a discharge velocity of the antibacterial fluid remains generally uniform during an entire period of discharge between the commencement of distribution and the termination of distribution.

8. A sanitizing device to provide for rendering at least of portion of bacteria on a hand of a person inert, the sanitizing device comprising:
a) a supply of antibacterial fluid capable of killing bacteria when brought into contact with the bacteria;
b) a first distribution nozzle having a path of distribution;
c) a second distribution nozzle having a path of distribution and wherein the path of distribution of the first distribution nozzle faces generally away from the path of distribution of the second distribution nozzle;
d) means to propel the antibacterial fluid through the first distribution nozzle and the second distribution nozzle;
e) activation means to commence distribution of the antibacterial fluid through the first distribution nozzle in the path of distribution and through the second distribution nozzle in the path of distribution wherein the hand of the person may have the antibacterial fluid sprayed thereon to kill at least a portion of bacteria on the hand of the person;
f) means to terminate distribution of the antibacterial fluid through the first distribution nozzle and through the second distribution nozzle.

9. The sanitizing device defined in claim 8 further comprising sensory means and blocking means, the sensory means to determine a presence of a non-child person as the user, the blocking means to prevent the commencement of distribution of the antibacterial fluid through the first distribution nozzle and the second distribution nozzle in the absence of the sensory means determining the presence of the non-child person as the user.

10. The sanitizing device defined in claim 9 wherein the sensory means further comprises a sensor positioned at an elevational height sufficient to ensure that the user has a height at least of a minimal measurement to ensure that the user is the non-child person.

11. The sanitizing device defined in claim 9 wherein the sensory means further comprises a heat detecting sensor capable of determining presence of a heat source at a predetermined location relative to the path of distribution of the first distribution nozzle wherein the heat detecting sensor may ensure that a trunk of the user is positioned at a height at least of a minimal measurement to ensure that the user is the non-child person.

12. The sanitizing device defined in claim 9 wherein the sensory means further comprises a weight determining sensor to ensure that the user is of a minimal weight to ensure that the user is the non-child person.

13. The sanitizing device defined in claim 8 wherein the first distribution nozzle and the second distribution nozzle are mounting on opposing sides of a panel.

14. The sanitizing device defined in claim 8 further comprising suction means and a first intake manifold and a second intake manifold, the suction means to draw in through the intake manifolds at least a portion of the antibacterial fluid propelled through the first distribution nozzle and the second distribution nozzle wherein the antibacterial fluid is generally prevented from leaving sanitizing areas of the sanitizing device.

15. A method of generally sanitizing a portion of an object, the method comprising the steps of:
a) providing a sanitizing device having:
1) a supply of antibacterial fluid capable of killing bacteria when brought into contact with the bacteria;
2) means to distribute the antibacterial fluid in a path of distribution in mist form;
3) means to commence the distribution of the antibacterial fluid;
4) means to terminate the distribution of the antibacterial fluid;
b) positioning the object relative to the sanitizing device;
c) positioning the portion of the object relative the sanitizing device in the path of distribution;
d) commencing the distribution of the antibacterial fluid wherein the antibacterial fluid is placed upon the portion of the object;
e) terminating the distribution of the antibacterial fluid.

16. The method defined in claim 15 wherein the means to commence further comprises a sensor capable of determining a presence in a zone of determination and wherein the method further comprises the step of moving the object relative to the zone of determination of the sensor.

17. The method defined in claim 15 wherein the object is a person and wherein the portion of the object is a hand of the person.

18. The method defined in claim 17 further comprising the step of rubbing the antibacterial fluid into the hands of the person following the commencement of the distribution of the antibacterial fluid to place the antibacterial fluid upon the hands of the person.

19. The method defined in claim 15 wherein the means to commence the distribution of the antibacterial fluid further comprises a sensor and wherein the step of commencing the distribution of the antibacterial fluid is activated by the step of positioning the portion of the object in the path of distribution wherein the sensor is activated.
